(19) United States
(12) Patent Application Publication
(10) Pub. No.: US 2013/0030234 A1
(43) Pub. Date: Jan. 31, 2013

(54) Single Module Apparatus for Production of Hydro-Carbons and Method of Synthesis

(71) Applicants: Viktor V. Astafiev, Kiev (UA); Sergii G. Iakovliev, Kiev (UA); Alexander Kozlov, Rockville, MD (US); Sergii A. Lytvynenko, Kiev (UA)

(72) Inventors: Viktor V. Astafiev, Kiev (UA); Sergii G. Iakovliev, Kiev (UA); Alexander Kozlov, Rockville, MD (US); Sergii A. Lytvynenko, Kiev (UA)

(73) Assignee: Alexander Kozlov, Rockville, MD (US)

(21) Appl. No.: 13/306,209

(22) Filed: Nov. 29, 2011

Related U.S. Application Data
(63) Continuation-in-part of application No. 13/191,854, filed on Jul. 27, 2011, now abandoned.

(51) Int. Cl. C07C 1/00; B01J 19/00

(52) U.S. Cl. 585/638; 422/198

(57) ABSTRACT

Disclosed herein is a method to synthesize hydrocarbons directly from water and atmospheric air in the presence of small amount of hydrocarbons. A module apparatus for gaseous and liquid hydrocarbons production and a technological process of hydrocarbons synthesis is provided. The peculiarity of the developed technological process is that atmospheric air and water are consumed in the process of synthesis, while a hydrocarbon matrix is maintained unconsumed.

The apparatus consists of a hydrocarbon synthesis chamber, a sump tank to collect hydro carbonic condensation derived in the process of synthesis, and a bubbling chamber. All of the chambers as well as sump tank are interconnected by means of pipes. The synthesis chamber is equipped with devices to supply water. Furthermore, the bumbling chamber is equipped with device to supply atmospheric air inside the chamber.

### FIELD OF THE DISCLOSED TECHNOLOGY

The presently disclosed technology is a method of direct synthesis of gaseous, gaseous-watery and liquid hydrocarbons on a module apparatus. The method comprises use of water and atmospheric (ambient) air, which are consumed during the synthesis process, as well as the use of hydrocarbons as an initial fill, which are maintained unconsumed through the technological cycle of the synthesis process (without external refill.)

### BACKGROUND

The existing hydrocarbon synthesis technologies, as a rule, are based upon the use of so-called synthesis gas or syngas (CO+H₂), from which various hydrocarbon compounds are obtained. The compounds are usually obtained at the presence of various catalysts under specific temperature and pressure or other conditions. See, e.g., U.S. Pat. No. 7,736,400 and Russian Patent 2062750.

Hence, the main energy expenditures are incurred during the preliminary stage of obtaining synthesis gas from various raw materials, such as fossils (coal) and charcoal. See, for example U.S. Pat. No. 7,459,594. The synthesis gas is derived through the process of pyrolysis of these substances, as exemplified in U.S. Pat. No. 7,758,663.

Technologies utilizing various wastes (petrochemical waste, bio-gasses from organic wastes, livestock waste, etc.) to produce consumable materials for further hydrocarbon synthesis require very high energy inputs as well. These high energy inputs required for decomposition of raw material (e.g. pyrolysis) are the main contributor to rendering the whole production process barely energy efficient. Thus, an alternative to the above can be the use of prime metabolic products: CO₂ and H₂O for production of synthesis gas for further synthesis of light hydrocarbons, (e.g. as disclosed in U.S. Patent Publications 2010/0022666, 2010/0022671, and 2011/0130474.) These prime metabolic products should include atmospheric air and various exhaust (burnt, oxidized gaseous products) gases as well. In this case it can be possible to bring close-loop technology up to industrial scale. Such technology is not only environmentally friendly but is autonomous, as it requires neither supply of raw material nor its thermal treatment.

The existing industrial hydrocarbon synthesis technologies utilizing water and atmospheric air are based upon creating conditions for water decomposition into hydrogen H₂, oxygen O₂, and extraction of carbon dioxide from ambient air.

One can relate to the above technologies, which utilize water electrolysis (e.g. Russian Patent 2213692) and accumulation of CO₂ from air (e.g. U.S. Pat. No. 7,427,368) within various chemical compounds at the presence of various catalytic agents with the use of plasma reactors (e.g. U.S. Pat. No. 7,867,457, and U.S. Pat. No. 6,853,142), et al. Then, obtained substances H₂, O₂, CO₂, as a rule, are brought to synthesis reactors, where specific temperature, pressure, presence of specific catalysts and so on are created, i.e. conditions that induce synthesis of CO+H₂ syngas, which serves as nuclei for subsequent synthesis of a variety of hydrocarbons. All these above methods and devices for hydrocarbons' synthesis from water and air require substantial amount of energy inputs, which in its turn renders final synthesis products expensive.

### OBJECTS OF THE DISCLOSED TECHNOLOGY

The inventors have been unable to locate a scientific or engineering solution (neither for method nor for technology) implemented in a working apparatus, which can synthesize hydrocarbons directly from water and atmospheric air in the presence of a hydrocarbon matrix, though such technologies exist in nature.

In a global system, where the Earth is a relative constant in terms of atmospheric make-up, the Earth's atmosphere can be viewed as being in a dynamic equilibrium between the processes of synthesis and breakdown of gases and compounds. Principal factors in the synthesis processes are biomass comprised of bacteria, plants, and animals, which, with the passage of time, disintegrate into gasses: vapor H₂O, nitrogen, oxygen and carbon dioxide and others. There are further reactions which take place between syntheses and de-synthesis cycles, and also produce various organic compounds such as paraffin, aromatics, naphthene etc. The most universal tool for forming such compounds is bacterial synthesis: it produces bio-gas (CH₄ with other compounds) and with assistance of so-called methane bacteria it produces ethyl alcohol, lactic acid souring products (bifidus and lacto bacteria), and butyric fermentation products (clostridial and other bacteria).

High-molecular paraffins such as wax and resin (for example: gum, oleoresin, coniferous trees' tar, caoutchouc rubber, resin) are formed as a result of plants' metabolism, and there are many others examples of heavy paraffins' production from the carbohydrate basis in the nature.

The initial tier of carbohydrates formation is photosynthesis:

CO₂+H₂O+hν=C₆H₁₂O₆ (I),

where “h” is Planck's constant, “ν” is green frequency of visible spectrum of Sun's radiation.
Formation of polysaccharides (cellulose, fructose, etc.) is in essence a polymerization reaction of the initial product (I). The general carbohydrates' structural formula is

C_{N}(H₂O)_{N} (II)

Where C₆H₁₂O₆+H₂O+enzymes→C_{N}(H₂O)_{N}(polysaccharides)+H₂O+enzymes→turn into→paraffins and olefins. Thus, the mixture of paraffins and olefins under influence of wide-spectrum radiation and slight heating gets ionized, and in contact with water gets hydrogenated. This leads to the formation, or in other words, to synthesis of the mixture of combustible hydrocarbons. Thus the paraffins are obtained from the compounds like (II) by the means of oxygen decoupling (complete or partial.)

Oxygen decoupling can be achieved either through thermal treatment in a corresponding medium, through bacterial treatment, or combinations thereof.

Polysaccharides (cellulose) subjected to initial bacterial fermentation and under subsequent thermal treatment can transform into paraffins. A bacterial synthesis gas transforms carbohydrates into paraffins. Structural formula (II) does not limit type of bond formed between water and carbon. In other words, there is a possibility of direct synthesis of paraffins through interaction of water vapor with carbon dioxide. Such interaction is possible only if reacting gases are ionized. Thus, it is necessary to bring two reacting gases (vapor and carbon dioxide) to excitation (metastable state). Under these conditions the very process of synthesis takes place, and paraffins and others hydrocarbon compounds can be formed. Thus, there are natural chemical reactions which produce hydrocarbons in the presence of a small quantity of the initial hydrocarbons (paraffins, olefins, ceresin, etc.). The initial hydrocarbons are considered the matrix, and notably the only consumables used for such synthesis are H₂O and CO₂ from atmospheric air.

### DEFINITIONS

Some terms used by the inventors through the text are defined as follows:

Small amounts of initial hydrocarbons which are put into the chemical synthesis chamber before the commencement of the work will hereinafter be referred to as “hydrocarbon fill” or “hydrocarbon matrix”. “Ether water” is a liquid derived from the process of synthesis, and in essence is a hydrocarbon condensation bound by oxygen. “Bubbling chamber” is a flask where uncondensed gases are derived during the process of synthesis, and are being caught and bound by water into water-gaseous solution. “Electric double layer” or “EDL” is a thin film consisting of two mutually phobic or non-wettable liquids located between the water and the boiling surface of the hydrocarbon fill. “Module” is a technologically complete cycle of operations realized on the apparatus.

### SUMMARY OF THE DISCLOSED TECHNOLOGY

Disclosed herein is a method to synthesize hydrocarbons directly from water and atmospheric air in the presence of small amount of hydrocarbons (hydrocarbon matrix) on a module apparatus and a technological process of gaseous and liquid hydrocarbons synthesis. The peculiarity of the developed technological process is that ambient air and water are consumables, while hydrocarbon matrix is technologically maintained unconsumed.

The apparatus consists of a hydrocarbon synthesis chamber, a sump tank to collect hydrocarbon condensation obtained in the process of synthesis, and a bubbling chamber. All chambers as well as the sump tank are interconnected by means of pipes. The synthesis chamber is equipped with devices to supply water, and the bumbling chamber is equipped with device to supply atmospheric air into the chamber.

The process of hydrocarbon synthesis takes place in the synthesis chamber, where the initial hydrocarbon fill has been placed. The hydrocarbons fill is heated up and brought to melted condition in the synthesis chamber, and then under very specific temperature, finely pulverized water is spray-injected through a nozzle into the synthesis chamber, and onto the boiling surface of the hydrocarbon fill. It shall be noted, that water is supplied periodically at equal intervals of time, at a specific temperature. Simultaneously with the water spray-injections into the synthesis chamber, air is supplied into the bubbling chamber.

As a result of water injections into the synthesis chamber where small amounts of initial hydrocarbon fill has been placed, and as a result of both the heating of the hydrocarbon fill and water injection, a steam-gaseous mixture forms. Then, due to colliding interaction of the finely pulverized water with the boiling surface of the hydrocarbon fill, the steam-gaseous mixture becomes ionized in the EDL. This in turn induces the commencement of adiabatic, plasma-chemical and exothermal reactions of synthesis, which produce a wide spectrum of synthesis gases: CO, H₂, O₂, CO₂, C₁-C₄, all in their metastable state. The gases then immediately react herewith and form synthesis-condensation of light hydrocarbons, ethers, carboxylic acids, spirits, etc. In order to maintain the balance of gases in the module apparatus a portion of both ether water and final product is returned to the synthesis chamber.

The present invention comprises a method of direct synthesis of the hydrocarbons on the module apparatus from such consumables as water and ambient air at the presence of non-consumable initial hydrocarbon fill and a module apparatus for production of gaseous, gaseous-watery and liquid hydrocarbons.

The disclosed technology is based upon chemical hydrocarbon synthesis, in a chamber that is in combination with a sump tank for collection of hydrocarbon condensation derived in the process of synthesis, and is also in combination with a bubbling chamber for collection of hydrocarbon gases obtained in the process of the synthesis. Together, the synthesis chamber and sump tank constitutes a technologically complete hydrocarbon synthesis module. The functional framework of the module apparatus reflects the main characteristics of the technological process of the hydrocarbon synthesis.

The upper inner parts of the hydrocarbon synthesis chamber, sump tank and bubbling chamber are inter-connected by a main pipe, while the sump tank in its lower inner part is connected with the synthesis chamber correspondingly by means of a branch pipe, which serves to direct synthesized gaseous-watery hydrocarbons mixture (ether water) from the sump tank to the synthesis chamber. The sump tank, at its inner mid-section portion, is connected with the synthesis chamber by means of a branch pipe, which serves to supply final liquid hydrocarbon product back to the hydrocarbon synthesis chamber in correspondence with the technological cycle. Furthermore, the bubbling chamber is connected by means of pipe to the device for supply of water to the synthesis chamber. The module apparatus is equipped with a device for air supply to the bubbling chamber.

The synthesis chamber is equipped with devices that use high-pressure spray nozzles for injection of water, ether water and final product into the working space of the synthesis chambers.

The synthesis chamber is equipped with a thermal device, which is installed inside of a tunnel going through the synthesis chamber, and which serves for heating of the hydrocarbon fill, as well as for heating and ionizing of the steam-gaseous mixture in the synthesis chamber. The thermal device is powered by an electric current source.

The thermal device is made, in an embodiment of the disclosed technology, of hard, refractory composite materials, sprayed-coated with fine-dispersion minerals and encased in protective jacket. The synthesis chamber is surrounded by a thin-dispersion loose-dry medium, which serves heat-stabilizing and heat-preserving purposes.

The present invention is further directed to synthesis of hydrocarbons directly from water and atmospheric air in the presence of the small amount of hydrocarbons without intermediate stage of production of H₂, O₂, CO₂, CO+H₂, CH₄ and other substances usually used for the synthesis of hydrocarbons.

The essence of the method of direct synthesis of the hydrocarbons on the module apparatus is based upon use of the hydrocarbon fill, which is placed inside the hydrocarbon synthesis chamber. The hydrocarbon fill is initially heated up and subsequently is brought to melted condition by means of the thermal device. After it is finely pulverized, water is spray-injected into the synthesis chamber onto the boiling surface of the hydrocarbon fill, while ambient air is supplied into the bubbling chamber.

The phenomenon is based upon creation of steam-gaseous medium, which in essence is a mixture of hydrocarbon gases and water steam. Upon the gases ionization, and water hydrolysis and ionization (when water is spray-injected upon the boiling surface of the hydrocarbon fill) an adiabatic, exothermal and plasma-chemical reaction is commenced within the mixture. However, there are few necessary conditions: high temperature gradients in the proximity of the boiling surface of the hydrocarbon fill, exothermal reaction (when water impacts against the surface of the hydrocarbon fill,) EDL resulting from non-wettability properties of two liquids (when water impacts against melted hydrocarbon fill), explosive cavitation resulting from water's impact against, and penetration into the melted hydrocarbon fill.

All the above listed conditions altogether cause ionization not only in the EDL but in the whole volume of the steam-gaseous mixture, and as a result free ions of H₂, O₂, CO, CO₂ and of such prime gases as C₁-C₄, C₅-C₁₀ appear all over the working space of the synthesis chamber. These above phenomena in their turn launch chemical reactions of the hydrocarbons' synthesis.

Thus, synthesis gasses CO+H₂, CH₄, etc. appear in a metastable condition within steam-gaseous mixture. Going at the presence of ambient air adiabatic and exothermal reactions, as a result of water impact against the boiling surface of the hydrocarbon fill, produce pressure spike in the synthesis chamber of 2-3 bars. But, the spike of pressure near droplets of water inside the boiling surface of the hydrocarbon fill reaches few dozen bars. Because of this, a portion of initial liquid hydrocarbons rises as a foam when a specific volume of water has been injected. Under the pressure the hydrocarbon gases derived in the process of synthesis enter the upper pipe connecting the synthesis chamber and the sump tank and start condensing as liquid, and eventually descending as liquid petroleum and ether water (hydrocarbon gases bonded with O₂) into the sump tank. After all the injected volume of water has reacted, the pressure in the synthesis chamber comes down, and ambient air enters into the chamber. The next injection of water begins the new cycle of the hydrocarbon synthesis. Thus, the synthesis progresses in a self-exited oscillatory mode. In order to maintain the balance of gases in the synthesis chamber as well as in the module apparatus and to maintain the density and mass of the initial hydrocarbons fills constant, ether water is returned by means of injections back onto the boiling surface of the hydrocarbon fill.

Thus, in the process of synthesis, the main consumable materials are water (e.g. tap) and CO₂ from ambient air, while the initial hydrocarbons fill remains non-consumed. Herewith, the amount of final synthesized product (e.g. petroleum and ether water) will not be lesser than the amount of injected water into the synthesis chamber.

### BRIEF DESCRIPTION OF THE DRAWINGS

The following detailed pictures render the functional framework and the evidences of the presented invention more understood:

FIG. 1 is the synthesis module apparatus diagram, which demonstrates the mode of operation, the technological process and the functional structure of the apparatus for synthesis of gaseous, water-gaseous and liquid hydrocarbons in correspondence with the embodiment of this invention.

FIG. 2 is schematic layout, which demonstrates the method and the process of the direct synthesis of the hydrocarbons from water and atmospheric air at the presence of the hydrocarbon matrix, which take place within the synthesis chamber of the module apparatus in correspondence with the embodiment of this invention.

FIG. 3 is schematic layout, which demonstrates mechanisms of steam-gaseous mixture ionization within the synthesis chamber of the module apparatus and the mechanisms which induce process of the hydrocarbons' synthesis in correspondence with the embodiment of this invention.

FIG. 4 comprises set of tables with comparative analysis of chromatograms of the conventional petroleum obtained from an oil refinery enterprise and the synthesis-petroleum obtained through invented by the authors technological synthesis process implemented on the module apparatus in correspondence with the embodiment of the invention.

### DETAILED DESCRIPTION OF EMBODIMENTS OF THE DISCLOSED TECHNOLOGY

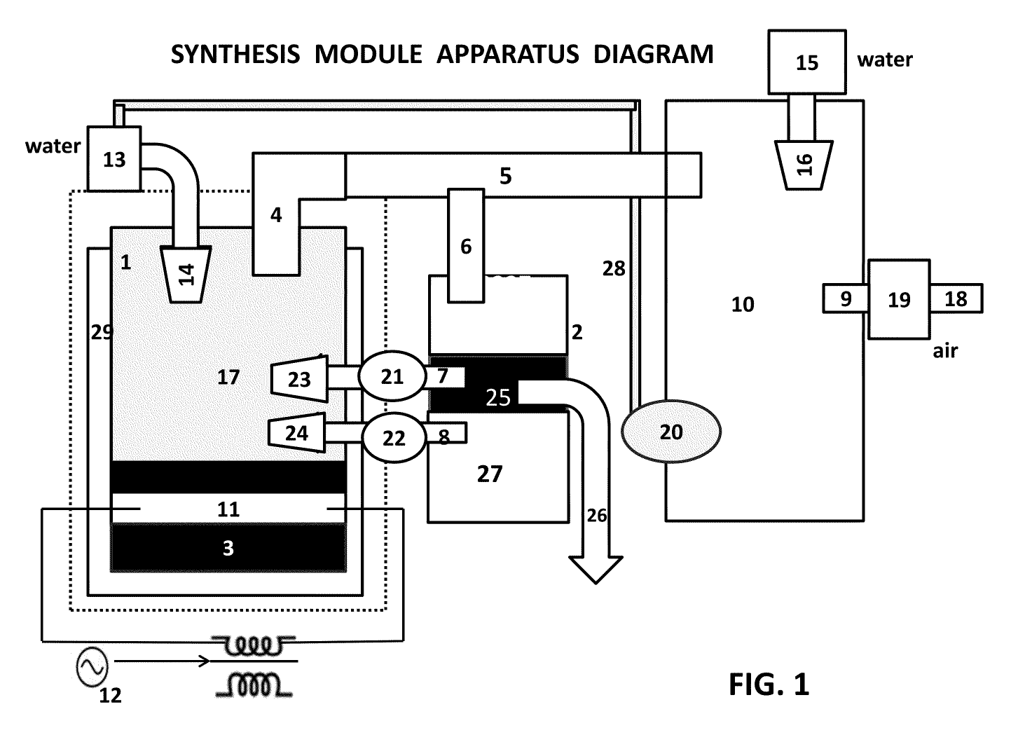

The process of synthesis is conducted in an automated module apparatus' synthesis chamber (FIG. 1) without any catalysts present or used. The module apparatus consists of a synthesis chamber, a sump tank, and a bubbling chamber. The apparatus is a single system with open-ended access of the ambient air, which enters the synthesis chamber via the bubbling chamber. At the initial stage the pressure inside the synthesis chamber is equal to the atmospheric pressure.

Before the commencement of operation, a hydrocarbon fill (matrix) is placed inside the synthesis chamber. Though the fill's composition can vary, in the conducted experiments the inventors used a composition which consisted of paraffin group hydrocarbons mixture containing: liquids from C₅H₁₆ to C₁₆H₃₄, gases varying from CH₄ to C₄H₁₀, and solids like C₁₇H₃₆. The fill's density in the conducted experiments was in the range 0.84 g/cm³ to 0.9 g/cm³. At the initial stage there was absolutely no water inside the synthesis chamber. In the conducted experiments the synthesis chamber has had a volume of 25 liters, while the initial hydrocarbon volume was from 7 to 7.5 liters.

The hydrocarbon fill is heated up by means of thermal element placed inside the tunnel. The thermal element extending all the way through the synthesis chamber. The process of heating should to be slow, and may start from 40° C. inside the synthesis chamber, increasing at an approximate pace of 2-3° C./min.

When the temperature inside the chamber reaches 50° C., appearance of the first droplets of light fraction hydrocarbons condensation may be present. This process corresponds to a straight-run refining of the initial hydrocarbon fill, where light portions of the fill evaporate.

Approximately 60 minutes from the beginning of heating, when the temperature inside the synthesis chamber reaches range of 117° C. to 120° C., the light flammable hydrocarbons' condensation appears inside the sump tank. Its mass is equal to about 5% of the initial hydrocarbon fill's mass.

When temperature inside the synthesis chamber reaches 117° C. to 120° C. the hydrocarbon fill comes to a simmering boiling state.

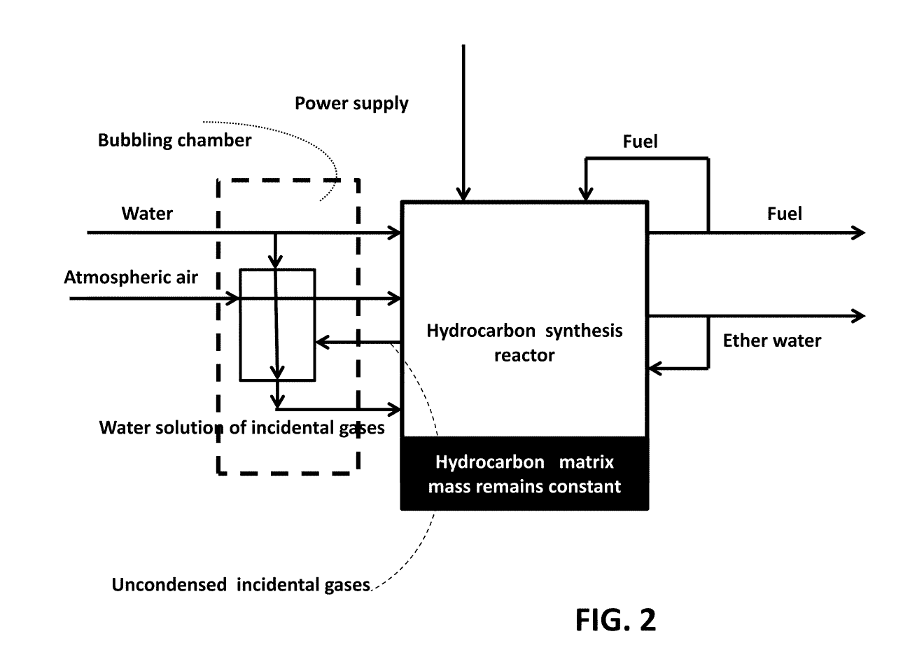

Hydrocarbon synthesis from water and ambient air stage at the presence of the hydrocarbon matrix is shown in FIG. 2.

When the temperature inside the synthesis chamber reaches 120° C. to 125° C., the first injection of finely pulverized water (12 ml) under high pressure through a nozzle inside the synthesis chamber is conducted for about 1.25 seconds.

The water injection's high degree of dispersion (size of the droplets) coming through the nozzle is an important element to the disclosed invention. In the conducted experiments of the direct hydrocarbon synthesis the degree of dispersion of water droplets has been from 30 to 40 microns under the pressure of 5 to 6 bars and from 10 to 15 microns under the pressure of 10 to 12 bars correspondingly. The velocity of the pulverized water jet has been no less than 50 to 60 m/sec.

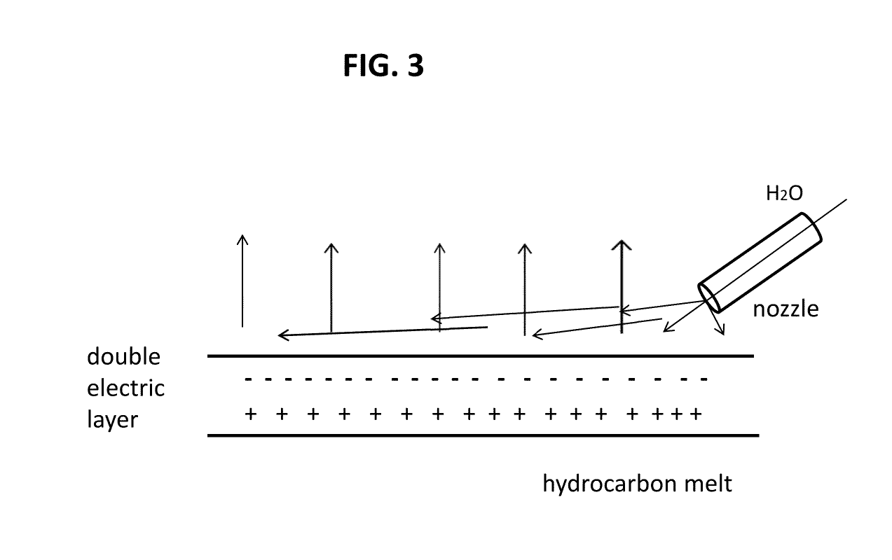

At this stage, due to the friction of the pulverized jet of water against the nozzle, droplets corning through the nozzle become electrified. This creates a certain voluminous electric charge. The level of frictional electrification depends upon the pressure and degree of dispersion. Water injection and water impact against the surface of the boiling hydrocarbon fill launches ionization process of the steam-gaseous mixture and commences the hydrocarbon synthesis process (see FIG. 3).

The method of low-temperature hydrocarbon synthesis is based upon a phenomenon of the steam-gaseous mixture's short adiabatic ionization time. The degree of ionization is determined by the speed of the transitional process, which takes place when highly pulverized water collides with boiling surface of the hydrocarbon fill. The collisional interaction of the highly pulverized water stream with the surface of the boiling hydrocarbon matrix on the verge of phase transition (interface) causes micro-explosive cavitation. This reaction has two main effects: a) a short-term (1 to 2 seconds) elevational rise of hydrocarbon fill in the synthesis chamber to about twice the level of the initial hydrocarbon fill before the commencement of operation, b) and a formation of steam-gaseous mixture of hydrocarbons within the working space of the synthesis chamber. The above affects are caused by the following: 1) Electrification of the water stream during the moment of pulverization due to friction against the nozzle [7, 8]; 2) High degree of residual electrification of the boiling hydrocarbon fill, namely by its electric characteristics [6]; 3) Appearance of an electric double layer (EDL) with high electric capacitance (10¹ to 10² micro F/cm²) and with high electrostatic intensity inside the EDL (10⁵ to 10⁶ V/cm) at the boundary (interface) between boiling surface of the hydrocarbon fill and finely pulverized water [9, 10, 11, 12, 13, 14]; 4). Cavitation vacuities (filled with steam) appear as a result of electro-kinetic processes as described above; and 5) The explosive nature of phase transition of electrified water droplets into gaseous state (steam.)

In general the effectiveness of the steam-gaseous mixture's ionization in the synthesis chamber is determined by the degree of polarization of two un-wettable liquids (boiling hydrocarbon fill and water), by the difference in their corresponding dielectric permittivity, and by the difference in temperature of injected water and of boiling hydrocarbon fill, that all above combined launches adiabatic ionization within the phase transition process.

Experimental data (collected by the inventors) permits quantitative evaluation of the steam-gaseous mixture's ionization degree based upon of material balance between the quantities of water and CO₂ injected into the synthesis chamber, and the quantity of synthesized products. Because balance of mass holds only under condition of very small injections of water, e.g. for the synthesis chamber volume of 25 liters the volume of water injection shall not exceed 2-3 mL, and then formula (I) holds:

MASS_{injected water}=MASS_{final product}+MASS_{ether water}

where MASS_{injected water} is mass of injected into the synthesis chamber water, MASS_{final product} is mass of synthesized final product, MASS_{ether water} is mass of ether water obtained in the process of synthesis. In this case, we neglect the quantity of incidental gases which have not condensed.

Thus, the degree of ionization is evaluated by the synthesized hydrocarbon mass' in correspondence with the above formula. Hence, the higher the degree of ionization corresponds to the higher the mass of final product and to the smaller the mass of ether water (semi-finished product.) Thus, the final products percentage of total synthesized products correlates to the degree of vapor-gaseous mixture in the synthesis chamber.

Data collected during numerous experiments shows that MASS_{final product} constitutes from 85 to 92% of MASS_{injected water}, MASS_{ether water} is from 6 to 10%, and MASS_{incidental gases} is from 2 to 5% correspondingly (neglected under the condition of small water injections). Such ratios point to a high degree of adiabatic ionization (from 85 to 92% correspondingly) achieved in the synthesis chamber in the process of synthesis of hydrocarbon products.

The subsequent water injections (and increased volume of injections up to 12 mL) make the process of synthesis more complex. Together with thr supply of air they bring about a number of phenomena, one being out-of-pile synthesis, which permits accumulation of an additional quantity of H₂CO₃ (due to humidification of CO₂ coming through the bubbling chamber, which is infused with water.) Then:

MASS_{injected water}+MASS_{carbon dioxide of ambient air}=MASS_{final product}+MASS_{ether water}+MASS_{out-of-pile synthesis products}.

where MASS_{injected water} is the mass of water injected into the synthesis chamber, MASS_{carbon dioxide of ambient air} is the mass of carbon dioxide contained in ambient air which came to the synthesis chamber, MASS_{final product} is the mass of synthesized final product, MASS_{ether water} is the mass of ether water obtained in the process of synthesis, and MASS_{out-of-pile synthesis products} is the mass of out-of-pile synthesis products, such as ether compounds (condensed and bound with water incidental gases), bound with H₂CO₃.

At the moment of water injection and EDL formation inside the synthesis chamber, an adiabatic reaction of newly generated steam takes place as a result of short-lived detonation and cavitation in the electrically charged droplets of water at the moment of their impact against the boiling surface of hydrocarbon fill. This causes the major portion of the steam to decompose into ions (hydrolysis): H₂O=H⁺+OH⁻. Simultaneously with hydrolysis, reactions of hydrogenation and electrification are taking place: coupling of hydrogen and hydroxyl with gases emitting from the hydrocarbon fill. As a result, the host of synthesis gases is formed inside the synthesis chamber within the steam-gaseous mixture, which further synthesizes the final product.

H⁺+OH⁻+CO⁺+O⁻+incidental gases(C₃H₈,C₄H₁₀ and others)+ionization, pressure→Synthesis→Final liquid product

Effectiveness of the synthesis is determined by few key factors, such as dispersion degree of injected water, excess pressure created by hydrocarbon gases emitting from the boiling hydrocarbon fill and appearance of electrified particles in near proximity to the boiling surface of hydrocarbon fill, which become nuclei of synthesis. Under 85% level of ionization the amount of ions from a single water injection reaches 85000 k (0.85 F, where F is Faraday constant: 99.5×10³ k.) The considerable amount of electrically charged hydrocarbon particles in the synthesis chamber form electrically charged medium with electrostatic intensity of 200 to 500V/m [1, 5, 11, 12, 13, 14]. Thus, the combination of electric charge of q=105 k/sec and velocity of 20 to 40 m/sec at which water is injected into the synthesis chamber produces impulse currents up to 10⁴ A, which in their turn (due to adiabatic nature of reaction) ionize water steam (H⁺+OH⁻) and turn it together with hydrocarbon gases (emitting from boiling hydrocarbon fill) into host of synthesis gases. Impact or collisional ionization accelerates straight-run refining of hydrocarbon fill into intensive process of direct synthesis of light hydrocarbons.

Carbon balance is determined by the so-called principal of equivalence existing between carbon content in ambient air (in dissipated state) and carbon content stored in carbohydrate biomass (solid state).

H₂O+CO₂Cₙ(H₂O)ₙ+bacterial decomposition→CO₂+H₂O

The above diagram shows correspondence of carbonic acid (H₂CO₃) and carbohydrate mass (as a main source of accumulated carbon). Various organic compounds, including carbohydrates, are products of this type of metabolic activity. Given the mechanism of hydrocarbon synthesis from water and ambient air, a large quantity of dust-like organic and mineral complex compounds must be taken into account (bacterial phytoplankton cells, pollen, micro fungus, organic waste products, increase concentration of various gases such as CO, CO₂, NO₂, NO, CH₄, etc.). Thus, concentration of CO₂ in the ambient air lying near the surface of land and bodies of water (especially in big cities) reaches from 0.5 to 1%, in contrast to widely accepted averaged number of 0.04%.

According to the present invention, hydrocarbon synthesis occurs when the pressure spikes at the moment of water injection. A portion of steam-gaseous mixture reaches an out-of-pile close diffusion zone, which expands proportionally to the number of injections and the duration of synthesis chemical reactions. This zone is characterized by increased content of ionized hydrocarbon gases. Due to the temperature difference (ΔT) between the close diffusion zone and remote diffusion zone (beyond air-supplying pipe) an out-of-pile convection (exchange of mass and heat) takes place in the pipes, in the bubbling chamber, and in the air-supplying device. The convection flow is proportionate to the difference between the temperature and humidity levels in these zones. The difference in absolute humidity is determined by quantity of steam coming from the synthesis chamber with the incidental gases into the diffusion zone. The difference in absolute humidity (Δn) is determined by the volume of a single water injection. The following equation determines the air circulation velocity from remote zone into the close one:

Vₐᵢᵣ=f(Δn,ΔT)

The above function of Vₐᵢᵣ is determined experimentally and contains coefficients, the values of which depend upon geometrical dimensions of the module apparatus in relation to air access, characteristics of thermal convection, wind, and air humidity and temperature outside the apparatus.

Fine droplets of water (in the steam-gaseous mixture) become nuclei of condensation and formation of carbonic acid CO₂+H₂O. The CO₂ enters with ambient air through convection exchange. The CO₂ accumulates in form of carbonic acid and ionized ether thin mist condensation in the diffusion zone, which returns to the synthesis chamber in a “breath-in” phase. Thus, ionized condensation in the close diffusion zone and air (CO₂) coming from remote diffusion zone constitute out-of-pile mass in the form of fine-drop ether condensation.

In the cross-section of the 1 m² diffusion zone the air velocity reaches 0.1 m/second. The general circulating air volume is about 360 m³ an hour, or in terms of carbonic acid is about 3-3.5 liters of fine-drop ether condensation (containing other ethers besides carbonic acid of various origins.)

Thus, carbonic acid H₂CO₃ concentration steadily increases in the process of the synthesis (proportionate to the number of water injections, and to the content of ionized water condensation. When the content of CO₂ depletes in the synthesis chamber, it gets replenished on the principal of communicating vessels from the bubbling chamber. This is based upon the inference that the concentration of carbon acid and incidental gases in out-of-pile zone facilitates maintaining the hydrocarbon fill's volume and composition at a constant level, and returning of the portion of final product and ether water (coming both from the sump tank and the bubbling chamber) back to the synthesis chamber.

Notably, carbon used in the process of hydrocarbon synthesis which initially came from light fractions of boiling hydrocarbon fill, is supplemented by the carbon dioxide of the ambient air, which subsequently becomes the main source of the carbon used in the hydrocarbon synthesis. The carbon dioxide enters the synthesis chamber through the bubbling chamber due to thermal convection, and together with fine condensation of water present in the ambient air it gets ionized and transformed into synthesis-gas:

CO₂+H₂O+^{ionization, pressure, temperature}=CO⁺+H⁺+OH⁻+O⁻

According to the present invention, which is based upon discovered phenomenon of steam-gaseous ionization caused by explosive cavitation of electrified fine droplets of water when they collide with the boiling surface of hydrocarbon fill, the direct hydrocarbons synthesis takes place at low-temperature (operational temperatures range from 120 to 180° C.).

From the point of view of physics, this process is analogous to vegetative biomass photosynthesis:

CO₂+H₂O+^{sunlight}→Cₙ(H₂O)ₙ

Earth atmosphere is constantly ionized due to sunlight. The density of light aero-ions in the proximity to the land surface averages from 400 to 500 ions/cm³. In other words, injected 1 M³ of air-gaseous mixture contains at least 10⁹ of ions, which in combination with ions of pulverized injected water, creates conditions for collisional ionization.

Under the condition of ionization within the synthesis chamber molecular chains of paraffin and ceresin compounds (contained in the hydrocarbon fill) become electrified, become so-called electrets, which can stay electrified for very long time [6]. The life time of electric charge within the mass of hydrocarbon fill can be from few days to few years. When this electric charge reaches specific level it increases charge density within the steam-gaseous mixture and its level of ionization.

Thus, as result of the first injection of pulverized water under high pressure and its collision with the surface of the boiling hydrocarbon fill at the moment of phase transition steam-gaseous ionized mixture forms. Simultaneously exothermal, plasma-chemical reactions of synthesis of the host of hydrocarbons are taking place. A portion of the ions are used for the final flammable product (e.g. petroleum) synthesis, the other portions of ions is used for synthesis of intermediate by-products such as gaseous hydrocarbons, carboxylic acid, spirits and ethers of these compounds (ether water).

At the moment of synthesis there is a 2-3 bar spike of pressure and 10-12° C. rise of temperature (based on experimental data), which lends itself to the exothermal nature of the ongoing reaction. The above process lasts 10⁻² to 10⁻³ seconds. After that, the final product of combustible liquid, as well as intermediate products such as gas-water solution (ether water) and synthesis gases are formed. When the pressure drops after 2-3 seconds, the ambient air enters the synthesis chamber through the bubbling chamber. Considering the residual electric charge within the hydrocarbon fill, portions of the carbon dioxide and water contained in the ambient air become ionized and partially replenish the hydrocarbon fill. In accordance with the developed technological chart this takes about 1 to 1.5 minutes.

The subsequent water injections through the nozzle into the synthesis chamber are performed at 1 minute intervals after the preceding injection. The iteration of the injections remains the same as above. The process of the synthesis is repeated. After the first injection cycle, 90% of the synthesized product is returned to the synthesis chamber. In all subsequent cycles up to 20% of the synthesized product is returned to the synthesis chamber. The purpose of this step is two-fold: to maintain the material balance and to restore the hydrocarbon fill's chemical composition with light hydrocarbon fraction range C₁-C₄.

The obtained ether water is periodically returned to the synthesis chamber through the pulverizing nozzle at following flow rate: about 15% to 20% of the obtained volume per every 20 water injections. The purpose of this operation is to maintain the material balance and to restore the hydrocarbon fill's chemical composition and density, which changes during the process of synthesis.

Furthermore, water from the bubbling chamber enriched with uncondensed incidental synthesized gases is periodically (once in every 10 injections) returned by means of a pump through the nozzle into the synthesis chamber. Each injection contains 30 mL and lasts approximately 1.5 seconds.

The operational range of temperature (135° C. to 153° C.) inside the synthesis chamber is maintained periodically by the thermal element intermittingly switching on for a period of 1 minute. All other times, the temperature inside the synthesis chamber during the process of the synthesis is maintained within the operational range due to adiabatic exothermal nature of the reactions taking place therein.

Thus, the technological cycle of the synthesis of the gaseous, water-gaseous and liquid hydrocarbons is cyclical and self-oscillatory. It comprises injections of water alternating with ambient air injection, and periodic returns of ether water and a portion of final product to the synthesis chamber.

During the process of hydrocarbon fill heating up (before reaching of the operational temperature) portion of molecular chains of the hydrocarbon fill gets destructed, so that part of the hydrocarbon fill's substance transforms into gaseous state. These are mainly homologues of hydrocarbon series (C₁-C₁₀), which together with ions of oxygen, hydrogen (plasma-gas), carbon oxide and hydroxyl synthesize the final liquid product (e.g. petroleum, under as stated above). Furthermore, collisional ionization (taking place when pulverized water jet impacts against the boiling surface of the hydrocarbon fill) causes explosive-cavitational destruction of long-molecular hydrocarbon compounds of C₃₀ series, what in its turn leads to formation of the host of synthesis gases (ether acids, ether spirits). Molecules with homologues C₃₀ decompose into gaseous fragments C₁-C₄, couple with hydroxyl OH, and correspondingly form either multi-atomic spirits or carboxylic acid.

Due to the involvement of the hydrocarbon fill in the chemical reaction changes occur in its chemical composition and its density. Experimental data has shown that after 50 water injections (without portion of final product and ether water returned into the synthesis chamber) the initial density of the hydrocarbon fill of 0.84 g/cm³ increased to 0.85 g/cm³. In time, during the process of direct synthesis the hydrocarbons fill's chemical composition becomes denser (due to light fraction de-enrichment) and its specific density increases.

Thus to enrich (replenish) hydrocarbon fill's chemical composition and to maintain technological process of synthesis stable, it is necessary to do the following: 1) Return to the synthesis chamber a certain portion of final product (in experiments 20-30% of the final product after 20 water injections) to maintain balance of liquids in the synthesis chamber; 2) Return to the synthesis chamber portion of synthesized products such as ether water containing dissolved incidental gases (as previously stated) to maintain the balance of incidental gases; 3) Supply an additional quantity of ambient air and carbon dioxide (CO₂) to the bubbling chamber by means of air supplying device between the water injections.

During the process of ionization long-molecular ionized compounds (electrets) of hydrocarbon fill, after cavitational detonation (beginning of the synthesis), adsorb ions of steam and carbon dioxide. Vacancies formed after explosive ionization are filled up by hydrocarbons contained in air, by the water-gaseous solution supplied from the bubbling chamber into the synthesis chamber, and by a portion of final synthesis-product (e.g. petroleum) and synthesized ether water supplied from the sump tank into the synthesis chamber. All the above together enable replenishment of the hydrocarbon fill and serve to maintain its the density and volume during the process of synthesis. They also serve to maintain composition of the steam-gaseous mixture in the synthesis chamber. Thus, during the technological cycle the hydrocarbon fill is maintained and remains non-consumed due to the infusions of water and ambient air (CO₂, H₂CO₃.)

Continuing, the chemical composition of the hydrocarbon fill becomes restored and enriched with carbon and hydrogen, and its initial density is restored as well. Thus, during the technological cycle the hydrocarbon fill remains non-consumed due to the infusions of water and atmospheric air (CO₂, H₂CO₃).

The main sources of the synthesis-products production are reactions of hydration, hydrogenation and etherification. These reactions take place in the EDL (as a result of collisional ionization following injections of finely pulverized water onto the boiling surface of the hydrocarbon fill), as well as in the whole volume of the steam-gaseous mixture:

Water injection+ionization,t° C.→H⁺+OH⁻ (III).

Supplied air (containing CO₂) and humid air (containing H₂CO₃) decomposes into:

CO₂+ionization,t° C.→CO⁺+O⁻H₂CO₃+hydrocarbon gases,ionization,t° C.→CO+H₂+hydrocarbon gases+O₂ (IV).

Reactions (III, IV) lead to alcohol acid formation. This is the main source of mass increase for carbon acids R—CO—OH formation; containing hydrocarbon's radical hydroxyl OH and carbon oxide CO. Alcohol acids are in essence esters, which form aromatic compounds that are present in synthesis-petroleum and other light hydrocarbons. Direct evidence of the reactions (III, IV) is numerous conducted experiments exhibit that when water is injected onto the surface of hydrocarbon fill, synthesis-petroleum is produced together with ether water and alcohol acids R—OH+R—CO—OH (VI). Furthermore, ions of hydrogen (III) under the condition of hydrogenation form paraffin, Isoparaffin and olefin, all of which become part of synthesis-products compound. Ionized gases CO⁺ and OH⁻ and H⁺ (i.e. plasma gas) are in metastable state with a lifetime of less than 10⁻³ seconds. Afterwards, they turn into more stable, intermediate synthesis gasses, and become part of steam-gaseous mixture. This steam-gaseous mixture is a main source of reproduction (including extended) of synthesis-products under the condition that H₂O and CO₂ and their derivatives (gaseous watery solution and air enriched with ether gases) served as the only consumable raw materials. (See FIG. 2).

Experiments to perfect parameters and frequency water injections onto the boiling surface of the hydrocarbon fill inside the hydrocarbon chamber, have demonstrated the following: 1) When a first injection of water was done in form of continuous jet or rough dispersion (size of droplets was over 70 microns), there was burst of steam and hydrocarbon fill rose from the synthesis chamber (density of water is higher than that of hydrocarbon fill) with an impulse pressure spike reaching few dozens of bars and temperature spike up to 300° C. due to exothermal reaction of water coming into contact with the hydrocarbon fill. The process of synthesis did not take place due to a burst of the hydrocarbon fill's substance outside the synthesis chamber and an absence ionization; 2) There was no synthesis while the temperature of the water injection was below 120° C.-125° C. Even if dispersion was 30-40 microns, there was small quantity of the light liquid product as a result of a straight-run refining of the hydrocarbon fill; and 3) Only if all conditions of the method developed by the inventors were met as described above, the synthesis took place and produced synthesis products (petroleum as in the experiments) and ether water.

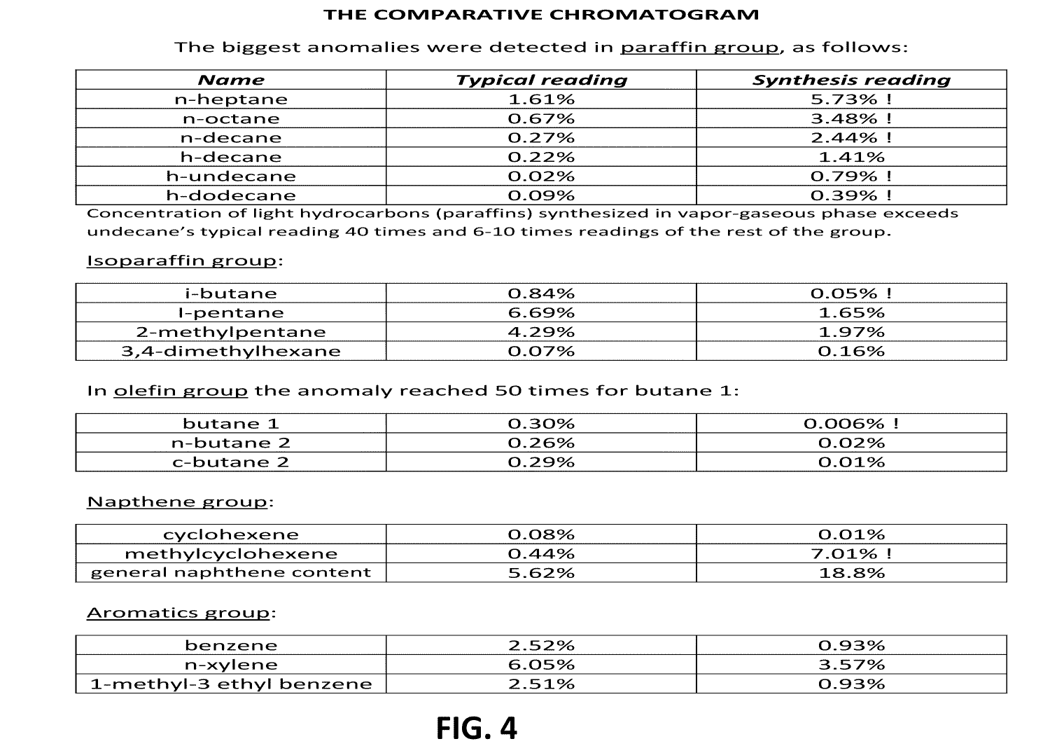

Based upon comparison of chromatograms of the synthesized petroleum and petroleum obtained through conventional pyrolysis, the main synthesis reactions taking place in the synthesis chamber (1) can be generalized and presented as follows:
R+H+OH+CO+O→synthesis gasses of the Paraffin group: (n-heptane, n-octane, n-decane, h-undecane, h-dodecane), plus synthesis gases of Isoparaffin group: (i-butane, i-pentane, 2-methylpentane, 3,4-dimethylhexane), plus synthesis gasses of Naphthene group: (cyclohexane, methyl-cyclohexane), plus synthesis gasses of aromatics' group (benzene, n-xylene, 1-methyl-3 ethyl-benzene.)
   The above listed gaseous compounds, which were obtained in the process of synthesis reaction after steam-gaseous mixture's condensation, induce generation of light liquid flammable hydrocarbons. The reactions described above are in essence of phenomenological type, and thus, take place in adiabatic regime in the presence of the exothermal phenomena. This is very conducive to and hereupon evokes synthesis phenomena, which utilizes wide range of intermediate metastable hydrocarbon compounds. Analysis of comparative chromatogram (FIG. 4) of chemical composition of petroleum obtained from oil-refinery conventional technology and chemical composition of petroleum obtained by the inventors on the apparatus (FIG. 5 Photo) shows the following: 1) the biggest differences in the composition were noted in light hydrocarbon compounds, which were synthesized in steam-vapor phase. These were namely paraffins, the reading of which was 40 times higher than that of conventional petroleum. The difference with the rest of the group was from 6 to 10 times; 2) For the Isoparaffin group, the difference in i-butane's reading was 13.5 times, and 3 to 10 times in other group components; 3) For the olefin group, the difference in butane 1 reading was 50 times, and the rest of the groups reading was between 10 to 20 times; 4) For the Napthene group, the difference in methyl-cyclohexene's reading was 16 times, and the rest of the group readings were between 4 to 10 times; and 5) For the aromatic group the difference in reading was from 2.5 to 3 times.

The material balance between water and carbon dioxide of ambient air on one side and synthesized products (final product, ether water and uncondensed incidental gases) on the other side (under the condition that hydrocarbon matrix' volume and density maintained constant) lends itself to the fact that the direct hydrocarbon synthesis from water and ambient air has been taking place. The method of low-temperature hydrocarbon synthesis was implemented on an automated module apparatus.

The apparatus as presented on the FIG. 1 and on the FIG. 5 (photo of the apparatus) comprises a hydrocarbon synthesis chamber (1), a sump tank (2) to collect hydrocarbon condensation derived in the process of synthesis, and a bubbling chamber (10). All chambers as well as sump tank are interconnected by means of pipes (4, 5, 6) in their upper parts correspondingly. The sump tank (2), at its middle portion is connected to the synthesis chamber by means of a pipe (7). The pipe (26) coming from the sump tank serves to shoe off the final synthesis product. The pipe (8) which connects the sump tank (2) with the synthesis chamber (1) at their lower portions serves to supply semi-finished by-product (i.e. ether water) back into the synthesis chamber. The synthesis chamber is equipped with devices (13) to inject water by means of nozzle (14), which is located in the upper part of the chamber (1).

The synthesis chamber (1) is also equipped with a device (22) to supply ether water through a nozzle (24). Another device (21) serves to periodically supply portion of the final product (25) through a nozzle (23) to the chamber (1).

The synthesis chamber (1) is equipped with a tunnel in which an arch-plasma thermal device (11) is installed. Atmospheric (ambient) air is supplied to the chamber (1) through the bubbling chamber, where air is enters by means of device (19) though pipes (9) and (18).

The bubbling chamber (10) is equipped with a device (15) to supply water through a nozzle (16). Another device (20) injects through a pipe (28) connected to the device (13) a water-gaseous solution.

A specific amount of hydrocarbons fill (3) is placed inside the chamber (1) before the commencement of the synthesis process. During the synthesis process inside the chamber (1), a steam-gaseous mixture (17) forms. The arch-plasma thermal device is powered by a source of electric current (12). The chamber (1) is surrounded by thin-dispersion loose-dry medium (29), which serves heat-stabilizing and heat-preserving purposes.

We claim:

1. A hydrocarbon synthesis method based upon a module apparatus, said method using hydrocarbon raw materials fill, which in the process of synthesis is maintained unconsumed;
said method also using consumables water and atmospheric air, wherein initially a hydrocarbon fill is placed in a synthesis chamber, is heated up by means of a thermal device, is brought subsequently to a melted state, and then finely pulverized water is injected through a nozzle into the synthesis chamber onto the boiling surface of the hydrocarbon fill, all while atmospheric air is supplied into a bubbling chamber.

2. The hydrocarbon synthesis method of claim 1, wherein water is spray-injected by means of a device through the nozzle into the synthesis chamber onto the boiling surface of the hydrocarbon fill periodically in equal intervals of time, under a specific temperature regime.

3. The hydrocarbon synthesis method of claim 2, wherein the atmospheric air is supplied into the bubbling chamber by means of an air-supplying device periodically, and alternatively with water spray-injections onto a boiling surface of the melted hydrocarbon fill in the synthesis chamber.

4. The hydrocarbon synthesis method of claim 3, wherein the steam-gaseous mixture that forms in the synthesis chamber after the hydrocarbon fill's heating and spray-injection of water, is ionized by the thermal device and by collisional interaction of injected finely pulverized water with the boiling surface of the hydrocarbon fill in the synthesis chamber.

5. The hydrocarbon synthesis method of claim 4, wherein the ionized steam-gaseous mixture is formed in the synthesis chamber as a product of opposing flows of gases flowing within a main pipe connecting the synthesis chamber and the bubbling chambers, into which water is periodically injected by the water-injecting devices, while ambient air is supplied into the bubbling chamber.

6. The hydrocarbon synthesis method of claim 5, wherein synthesized hydrocarbon products resulting from chemical reactions taking place in the synthesis chamber condense in the main pipe, the main pipe being where a final product accumulates and further connecting the synthesis chamber and the bubbling chamber with a sump tank.

7. The hydrocarbon synthesis method of the claim 6, wherein a hydrocarbon final product and ether water collected in the sump tank liquid in unequal parts of different densities are both periodically, alternatively infused by means of devices through nozzles correspondingly back into the synthesis chamber for sustaining synthesis chemical reactions within the synthesis chamber as well as for maintaining the hydrocarbon fill's density and volume at constant levels.

8. The hydrocarbon synthesis method of the claim 7, wherein water and ether water are alternatively spray-injected into the synthesis chamber.

9. The hydrocarbon synthesis method of claim 8, wherein atmospheric air is supplied into the bubbling chamber simultaneously with injections of water into the bubbling chamber, while water-gas solution from the bubbling chamber is supplied by means of a water-siphoning device through a connecting pipe to the water supplying device of the synthesis chamber for injections into the synthesis chamber.

10. The hydrocarbon synthesis method of claim 9, wherein semi-processed by-products are obtained during the process of synthesis, after condensation reaches the sump tank, and after the final liquid product is taken out; said by-products being completely recycled back into the synthesis chamber for further synthesis into the final products.

11. The hydrocarbon synthesis method of claim 10, wherein after the process reaches a specific temperature regime, the temperature within the synthesis chamber is sustained by exothermic reactions that result from finely pulverized injected water colliding against the boiling surface of the hydrocarbon fill, thus permitting the process of synthesis to continue with the thermal device intermittently being switched off and on for specific times intervals.

12. A module apparatus for direct synthesis of gaseous, gaseous-watery and liquid hydrocarbons comprising:
a chemical synthesis chamber equipped with a tunnel with a thermal device placed therein;
an electric current source for powering the-thermal device;
a sump tank for collecting hydrocarbon condensation during the synthesis process;
a bubbling chamber; and
the thermal device being configured for heating a hydrocarbon fill in the synthesis chamber, and heating and ionizing a steam-gaseous mixture in the synthesis chamber.

13. The module apparatus of the claim 12, wherein the sump tank and the bubbling chamber are connected within the synthesis chamber by a main pipe;
the sump tank being connected to the synthesis chamber by a first branch pipe, the first branch pump serving to direct a synthesized gaseous-watery hydrocarbon mixture from the sump tank to the hydrocarbon synthesis chamber; and
the sump tank also being connected to the synthesis chamber by means of a second branch pipe, the second branch pipe serving to supply a portion of the liquid hydrocarbon condensation back into the synthesis chamber.

14. The module apparatus of claim 13, wherein the bubbling chamber is equipped with a first device with a nozzle for injecting water into the bubbling chamber to bind with uncondensed gases; the bubbling chamber also equipped with a second device to siphon off the water-gaseous solution; the second device being connected by a pipe with a water supply device for injecting water into the synthesis chamber.

15. The module apparatus of claim 14, wherein the bubbling chamber is equipped with a device for supplying atmospheric air into the synthesis chamber.

16. The module apparatus of claim 15, wherein the synthesis chamber is equipped with a device comprising a high-pressure nozzle for finely pulverized water injection into the working space of the synthesis chamber;
the synthesis chamber also being equipped with a second device comprising a high-pressure nozzle for finely pulverized ether water injection into the working space of the synthesis chamber; and
the synthesis chamber further being equipped with a third device comprising a nozzle for injection of a portion of a final product back into the synthesis chamber.

17. The module apparatus of claim 12, wherein the thermal device is made of hard, refractory composite materials, sprayed-coated with fine-dispersion minerals and is encased in a protective jacket.

18. The module apparatus of claim 12, wherein the synthesis chamber is surrounded by a thin-dispersion loose-dry medium; said medium serving for heat-stabilization and heat-retention.
